(19) United States
(12) Patent Application Publication
(10) Pub. No.: US 2024/0150718 A1
(43) Pub. Date: May 9, 2024

(54) METHOD FOR GENE REPAIR IN PRIMARY HUMAN MUSCLE STEM CELLS (SATELLITE CELLS) IN VITRO AND GENETICALLY REPAIRED HUMAN MUSCLE STEM CELL

(71) Applicants: CHARITÉ - UNIVERSITAETSMEDIZIN BERLIN, Berlin (DE); MAX-DELBRÜCK-CENTRUM FÜR MOLEKULARE MEDIZIN, Berlin (DE)

(72) Inventors: Simone SPULER, Berlin (DE); Helena ESCOBAR, Berlin (DE); Christian STADELMANN, Berlin (DE)

(73) Assignees: CHARITÉ - UNIVERSITAETSMEDIZIN BERLIN, Berlin (DE); MAX-DELBRÜCK-CENTRUM FÜR MOLEKULARE MEDIZIN, Berlin (DE)

(21) Appl. No.: 18/280,154

(22) PCT Filed: Jan. 31, 2022
(86) PCT No.: PCT/EP2022/052214

§ 371 Date: Sep. 1, 2023

(30) Foreign Application Priority Data
Mar. 4, 2021 (EP) 21160696.7

(51) Int. Cl. C12N 5/077; C12N 9/22; C12N 15/11; C12N 15/85; C12N 15/90

(52) U.S. Cl. CPC C12N 5/0659; C12N 9/22; C12N 15/11; C12N 15/85; C12N 15/90; C12N 2310/20; C12N 2320/34; C12N 2800/107

(57) ABSTRACT

It is provided a method for gene repair in primary human muscle stem cells (satellite cells) in vitro comprising the following steps: providing a sample of an isolated muscle-fiber containing tissue sample collected from at least one patient with a monogenic muscle disease, wherein the monogenic muscle disease is caused by at least one mutation in at least one gene encoding for at least one muscle protein; isolating and cultivating primary stem cells from said muscle-fiber containing tissue sample, and correcting the at least one mutation in the at least one gene encoding for at least one muscle protein in the cultivated primary stem cells by targeted modification of the at least one mutation by gene editing using CRISPR/Cas-based tools.

### CROSS-REFERENCE TO A RELATED APPLICATION

This application is a National Phase Patent Application of International Patent Application Number PCT/EP2022/052214, filed on Jan. 31, 2022, which claims priority of European Patent Application Number 21 160 696.7, filed on Mar. 4, 2021.

### BACKGROUND

The disclosure relates to a method for gene repair in primary human muscle stem cells (satellite cells) in vitro/ex vivo and genetically repaired human muscle stem cell obtained by using said method.

Muscle fibers are syncytial structures with postmitotic nuclei formed by the fusion of myogenic progenitor cells, called myoblasts, during prenatal and postnatal development. Skeletal muscle can regenerate from muscle stem cells (MuSC), also called satellite cells, a pool of tissue-specific stem cells located between the muscle fiber membrane (sarcolemma) and the basal lamina that surrounds every fiber (Mauro A. SATELLITE CELL OF SKELETAL MUSCLE FIBERS. J Biophys Biochem Cytol. 1961; 9(2):493-495). In healthy muscle, satellite cells are quiescent or slow cycling. When activated in response to severe damage, they extensively proliferate and give rise to large numbers of myoblasts that fuse to damaged myofibers or to one another to generate new myofibers (Yin H, Price F, Rudnicki M A. Satellite Cells and the Muscle Stem Cell Niche. Physiol Rev. 2013; 93(1):23-67). Skeletal muscle regeneration cannot occur without satellite cells. Patients with muscular dystrophy (MD) suffer constant tissue degeneration, which prompts satellite cells to be constantly activated, leading to satellite cell exhaustion, regenerative deficit, and replacement of muscle by fat and connective tissue (Blau H M, Webster C, Pavlath G K. Defective myoblasts identified in Duchenne muscular dystrophy. Proc Natl Acad Sci USA. 1983; 80(15):4856-4860).

Cell replacement therapies with well-defined and highly myogenic cell populations could represent a safe and long-term treatment avenue for MD patients, however MuSC are scarce and traditionally difficult to manipulate ex vivo. In addition, skeletal muscle is the most abundant tissue in the body, thus developing cell replacement therapies for MD patients poses substantial challenges.

Various methods have been developed to produce new muscle cells to use in cell replacement therapies for MD. Allogeneic transplantation of myoblasts or mesangioblasts have so far failed to reach any clinical benefit. Induced pluripotent stem cells (iPSC) are an attractive and unlimited source of healthy cells and several protocols are in place to differentiate them into transplantable myogenic progenitor-like cells. However, iPSC-derived muscle cells do not yet meet quality and safety criteria for transplantation into patients. Overall, allogeneic transplantation requires immunosuppression or cloaking of transplantable cells, adding another level of uncertainty, and has not yet been shown for iPSC-derived muscle cells.

Autologous transplantation of primary muscle stem cells (MuSC) would be more predictable.

It has been previously shown that MuSC can be isolated and substantially expanded from human muscle biopsy specimens, and that they maintain in vivo regenerative capacity in xenograft models (Marg A, et al. Human satellite cells have regenerative capacity and are genetically manipulable. J Clin Invest. 2014; 124(10):4257-4265). Using them in an autologous setting for MD patients would require correcting the genetic defect before reimplantation. A genetically corrected muscle stem cell would be very similar to healthy (“wild-type”) muscle stem cells, but not identical. Formally, this has been demonstrated in base-edited human fibroblasts carrying a mutation in LMNA (Koblan et al., In vivo base editing rescues Hutchinson-Gilford progeria in mice. Nature 2021; doi.org/10.1038/s41586-020-03086-7, FIG. 2d). Healthy MuSC from the patients do not exist (as they all carry the genetic defect), hence the need for gene repair in a therapeutic context.

Muscular dystrophies (MD) are >40 monogenic diseases leading to severe muscular debility. No curative treatment is at present available. Skeletal muscle can regenerate from muscle stem cells and their myogenic precursor cell progeny, myoblasts.

For MD, there is presently no treatment or cure. However, there are ample activities to change this. Companies and research groups are intensively working on solutions. Exon skipping strategies, gene replacement approaches using cDNAs packaged in an AAV vector are in clinical trial stage for some MD. Cell-based therapies for MD are realistic and probably soon available (Bessetti 2020 J. Clin. Invest.). Cell-based therapy approaches can rely either on a “universal donor cell” which lacks individual immunological markers or autologous cells that are “genetically repaired” and transplanted back into the patient.

CRISPR/Cas systems allow for unprecedented precision and relative ease in targeting defined regions of the genome. Since the original description of Cas9 as an RNA-guided endonuclease, numerous directed evolution and mutagenesis approaches have resulted in Cas enzymes with enhanced specificity, and thus increased safety with respect to on-versus off-target editing profiles. In addition, engineered Cas9-fusion proteins like base and prime editors have enabled highly precise re-writing of the genome independent of cellular DNA repair pathway, cell cycle phase or an exogenous DNA template. One example is adenine-base editors (ABE), which convert A to G nucleotides at target genomic loci by combining the RNA-guided DNA binding capacity of Cas9 with the enzymatic activity of an evolved tRNA adenosine deaminase.

Chemmello et al. (Correction of muscular dystrophies by GRISPR gene editing, J. Clin. Investigation, 2020, 130: 2766-2776) provides an overview of application of CRISPR/Cas systems for correcting muscular dystrophies. Different strategies are described. A) Exon deletion by double cut myoediting: Here a mutated exon is excised to yield a truncated, but functional dystrophin. This strategy is effective for deleting exons that encode regions of dystrophin that tolerate deletions. However, exon deletion can generate diverse and unpredictable genome modifications, including exogenous DNA integration or aberrant splicing at the cut sites. B) Exon skipping and exon reframing by single cut myoediting for restoring the correct ORF: Only one sgRNA is used to generate a single cut in the genome. The sgRNA is designed to target a genomic sequence in the close vicinity of the intron-exon region of the out-of-frame exon and to produce one DSB. An alternative strategy for inducing exon skipping with antisense oligonucleotides without modifying the genome exists and is potentially safe, but can only be used to treat a subset of patients with a defined type of mutations in genes like DMD, where removing one or several exons can be of theoretical benefit, and needs periodic administration. C) Nucleotide myoediting for correcting point mutations: This allows for site-specific single nucleotide replacement.

Chemmello et al. also provides an overview of possible In Vivo delivery methods for the CRISPR gene editing system. This includes engineered nanoparticles, muscle progenitor engraftment and AAV delivery,

In vivo gene supplementation therapy using adeno-associated viral vectors (AAV) may be suitable for genes up to a certain size. However, the exogenously provided coding sequence is not physiologically regulated in terms of splicing or spaciotemporal expression and poses a risk of insertional mutagenesis. AAV-mediated CRISPR/Cas9 delivery directly into the muscle has enabled highly efficient in vivo gene editing in Duchenne's muscular dystrophy (DMD) mice and large animal models and could potentially reach and permanently repair a very large fraction of myonuclei. In vivo adenine base editing (ABE) in muscle through AAV delivery has been achieved in a DMD mouse model. However, if not done before substantial degeneration, fatty-fibrous replacement and muscle stem cell exhaustion have occurred, the disease course may not be reversible by either treatment.

In addition, many people have pre-existing antibodies against AAV, Cas9 or other Cas proteins, which interfere with in vivo delivery of either of them. Even in patients with no pre-existing immunity, repeated administrations of AAV (for gene supplementation therapy) or AAV-Cas9 (for in vivo gene editing) would not be possible due to adaptive immunity against the virus or the bacterial Cas9 protein triggered by the first administration. This may be especially critical for gene supplementation therapy with AAV vectors, since skeletal muscle tissue has a constant turnover (which is much faster in dystrophic muscle) and thus long-term transgene expression would be hindered by the gradual loss of AAV genomes. Toxicity of systemic AAV administration has also resulted in the death of three patients in a clinical trial of myotubular myopathy due to liver-related adverse events. In two other trials for spinal muscular atrophy (SMA) and DMD, adverse events resulted from an immune response to AAV.

Indeed, sustained Cas9 expression following AAV-mediated CRISPR/Cas9 delivery has been shown to induce muscle inflammation as well as anti-Cas9 humoral and cellular immune responses in dogs. This appears to be specific to Cas9 and may represent a critical barrier for AAV-CRISPR therapy in large mammals (Hakim et al., Nat Commun. 2021 Nov. 24; 12(1):6769. doi: 10.1038/s41467-021-26830-7.)

Non-targeted insertion of therapeutic transgenes via e.g. transposon or integrating viral vectors poses risks of insertional mutagenesis due to uncontrolled insertion sites and adds non-endogenous sequences flanking the transgene, which are needed for the genomic integration process.

In vivo gene editing interventions have shown success in repairing the genetic defect in postmitotic myonuclei within muscle fibers in mice and dogs (Amoasii 2018 Science, doi: 10.1 126/science.aau1549), and pigs (Moretti 2020 Nat Med, doi: 10.1038/s41591-019-0738-2), but reaching and repairing MuSC remains a significant challenge for approaches to deliver the gene editing machinery in vivo. Moreover, it is well known that in muscular dystrophies, skeletal muscle is progressively replaced by fat and connective tissue, losing the capacity to regenerate due, amongst others to MuSC exhaustion. Thus, in vivo gene editing may not suffice for a long-term therapeutic effect because skeletal muscle has high turnover, especially in muscular dystrophy, and sustained healthy muscle homeostasis thus requires a healthy pool of MuSC.

As can be seen from the above, the presently available approaches for gene repair of muscle cells and tissue are still limited.

### SUMMARY

Thus, there is a need for an alternative approach for genetic repair of muscle cells that can be used for cell replacement therapies without having the disadvantages described above. It would be of particular advantage to provide an effective method for the genetic repair of autologous primary muscle stem cells of a patient with muscular dystrophy.

This object is solved by a method having features as described herein.

Accordingly, an ex vivo (in vitro) method for gene repair in primary human muscle stem cells (satellite cells) is provided, wherein the method comprises the following steps:
providing a sample of an isolated muscle-fiber containing tissue specimen collected from at least one patient with a monogenic muscle disease, wherein the monogenic muscle disease is caused by at least one mutation in at least one gene encoding for at least one muscle protein;
isolating and cultivating primary muscle stem cells from said muscle-fiber containing tissue sample, and
correcting the at least one mutation in the at least one gene encoding for at least one muscle protein in the cultivated primary muscle stem cells by targeted modification of the at least one mutation by gene editing using CRISPR/Cas-based tools,
wherein the CRISPR/Cas-based gene editing tools are delivered to the cultivated primary muscle stem cells by at least one DNA and/or RNA based carrier, in particular plasmids, non-integrating viral vectors, mRNA or protein.

The present targeted therapeutic method by means of CRISPR/Cas-based, in particular CRISPR/Cas9-based gene/base editing techniques uses primary human myoblasts with regeneration potential which are isolated and collected according to a previously described method (WO 2016/030371 A1). Primary human myoblasts collected by this method are >98% pure and therefore accessible for gene editing.

Gene delivery studies previously performed on primary human myoblasts using a transposase system (WO 2016/030371 A1) also illustrated that the isolated primary human muscle stem cells are generally susceptible for genetic manipulation. However, transposase mediated gene insertions are non-targeted and thus not a preferred delivery tool for site-specific mutations.

In contrast, by using now CRISPR/Cas based tools specific mutations can be corrected and gene edited primary human myoblasts with regenerative capacity are provided.

This was not to be predicted. In particular, in the past it was commonly thought that human muscle stem cells could not be transfected and/or genetically manipulated very efficiently. This would hamper any approaches to use human muscle stem cells for therapy because, unlike for example iPS cells, primary MuSC cannot be expanded from one single gene-repaired clone to produce sufficient numbers of cells for e.g. an autologous graft. That means that obtaining a high enough % of cells carrying the desired edit at the population level is essential. For these reasons, gene editing in primary human MuSC was not attempted previously.

In Marg et al. (J. Clin. Invest; 2014, 124:4257-4265) it was shown that only a few green muscle cells transfected inefficiently with lipofectamine and with a plasmid vector encoding GFP. Based on these results, it was not predictable that targeted gene correction would be able to correct primary ex vivo expanded human MuSC with efficiencies that go beyond 90% for some of the target loci/mutations.

Chemello et at. (J. Clin. Invest.; 2020, 130:2766-2776) mentions that muscle progenitor cells were corrected by CRISPR genome editing and then engrafted in muscle of mdx mice by intramuscular injection as shown by Zhu et al. (2017 Mol Ther Nuc Acids, https://doi.org/10.1016/j.omtn.2017.02.007). Here, Cas9 was delivered ex vivo into MuSC from a dystrophic mouse using adenovirus. However, editing efficiency of the mouse MuSC appears to be low (FIG. 2E) and no accurate quantification is shown. Furthermore, said approach includes a low efficiency of engraftment and the inability to generate muscle stem cells with long-term repopulation potential.

In contrast, here a highly efficient targeted genome editing in human primary MuSC at the population level using plasmid-based transfection is shown for the first time. With the present method, >90% edited MuSC with an FACS-based enrichment step to select for transfected cells and 40-60% efficient editing without that enrichment step are obtained. It is shown that human primary MuSC can undergo gene editing with very high efficiency using different methods to deliver the gene editing machinery ex vivo (mRNA nucleofection), which is much more advanced in terms of clinical translation and that doesn't require the use of a selection marker to enrich for gene edited cells.

The transplanted autologous myoblasts in which a phenotypic defect has been therapeutically addressed by gene editing techniques in a site-specific manner can build new muscle tissue in which the pathological phenotype is rescued totally or partially.

Furthermore, these cells are able to repopulate the recipient muscle with muscle stem cells that would sustain muscle homeostasis and regeneration long-term. It was not obvious that primary human MuSC that have undergone such a targeted genetic manipulation ex vivo would retain their regenerative properties and be able to not only build new muscle fibers in vivo following transplantation, but also reconstitute the stem cell compartment.

The present method has several advantages: The cells can be applied in an autologous setting, in which they are unlikely to trigger immune rejection or adverse immune reactions. The edited cells maintain their ability to produce new muscle fibers and repopulate the muscle stem cell pool in vivo, thus it is probable that a single administration suffices for a long-term, sustained therapeutic effect. Skeletal muscle tissue has a very low propensity to develop tumors, and so do muscle stem cells and myoblasts. The edited cells can be thoroughly checked for off-target events and biosafety profile prior to reimplantation. These cells are therefore transplantable in an autologous setting with a small and calculable risk.

In an embodiment of the proposed solution, the monogenic muscle disease comprises one of the following: muscular dystrophy (MD) including all types of limb-girdle muscular dystrophy (LGMD), in particular of type LGMD1/D, LGMD2/R (Straub et al., 2018; https://doi.org/10.1016/j.nmd.2018.05.007), all X-linked muscular dystrophies (Emery-Dreyfuss MD, Duchenne MD, Becker MD), and all MDs caused by repeat expansion (i.e. myotonic dystrophy type 1 and type 2) or repeat deletion (facioscapulohumeral muscular dystrophy) mutations. Pax7 myopathy, a very rare disease (Marg et al., Nat Commun. 2019 Dec. 18; 10(1):5776. doi: 10.1038/s41467-019-13650-z), and VCP myopathy, are included as well.

It is to be understood that the at least one gene mutation in the at least one gene encoding for one muscle protein can be a deletion, insertion, point mutation, repeat expansion, or repeat deletion, in particular a deletion or point mutation. In a preferred embodiment, only monogenic diseases are considered with one mutation on each allele. The mutation might be the same (homozygous) or different (compound heterozygous).

As mentioned above, precise and efficient gene repair in primary somatic stem and progenitor cells ex vivo is increasingly plausible due to the rapid development of CRISPR/Cas-based tools for gene editing, some of which are independent from the cellular DNA repair pathway choice.

Said CRISPR/Cas-based tools, in particular CRISPR/Cas9-based tools, may be used for at least one of the following gene editing approaches: base editing, in particular adenine base editing (ABE), cytidine base editing (CBE), C-to-G base editing (CGBE), glycosylase base editing (GBE), prime editing, non-homologous end joining (NHEJ), microhomology-mediated end joining (MMEJ) and/or homology-directed repair (HDR).

Base editors are RNA-programmable deaminases that enable precise single-base conversions in DNA or RNA. Base editors do not create double-stranded DNA breaks and therefore minimize the formation of undesired editing byproducts, including insertions, deletions, translocations, and other large-scale chromosomal rearrangements.

Adenine base editing (ABE) enables the precise targeted conversion of adenine into guanine nucleotides without inducing DNA double-strand breaks (Gaudelli N M, et al. Programmable base editing of A⋅T to G⋅C in genomic DNA without DNA cleavage. Nature. 2017; 551(7681):464-471). An ABE consists of a catalytically impaired Cas9 in fusion with an adenine deaminase enzyme (TadA) that converts adenine into inosine on the single stranded DNA bubble created by Cas9 binding to a target site. Inosine is subsequently replaced by guanine. To be accessible to the deaminase, the target adenine must be located at a defined distance from the protospacer adjacent motif (PAM), the so-called ABE activity window. Because of its predictable outcome, high precision and reduced off-target effects (20, 21), ABE is a safe gene editing tool.

Cytosine base editors (CBEs or BEs) enable efficient, programmable reversion of T⋅A to C⋅G point mutations in the human genome. BEs are comprised of a cytidine deaminase fused to an impaired form of Cas9 (D10A nickase) tethered to one (BE3) or two (BE4) monomers of uracil glycosylase inhibitor (UGI). This architecture of BEs enables the conversion of C⋅G base pairs to T⋅A base pair in human genomic DNA, through the formation of a uracil intermediate.

More recently, base editors have been developed (Pin-point™) in which the effector domain (deaminase) is not fused to Cas9. Instead, the effector is fused to a cognate ligand for an RNA aptamer that is included in the gRNA, thereby facilitating the recruitment of the effector to the Cas9/gRNA complex (Collantes et al., 2021, CRISPR J, DOI: 10.1089/crispr.2020.0035)

Non-homologous end joining (NHEJ) repairs double-strand breaks (DSB) in DNA (for example, generated by Cas9 cutting), often introduceing small insertions and/or deletions (indels) at the repair site.

Homology directed repair (HDR) pathway makes use of a provided DNA template with regions of homology to the target site, hence allowing for the generation of desired alterations.

In a more specific embodiment, the at least one mutation is located in at least one of the following genes: LMNA encoding for lamin A/C, CAPN3 encoding for calpain 3, DYSF encoding for dysferlin, SGCA encoding for α-sarcoglycan, VCP encoding for valosin containing protein, PAX7 encoding for paired box 7, NCAM1 encoding for neural cell adhesion molecule 1, DMD encoding for dystrophin.

α-Sarcoglycan is a 50 kDa transmembrane protein, part of the sarcoglycan complex and the dystrophin-associated protein complex (DAPC). The DAPC protects muscle fibers from mechanical stress and its dysfunction leads to various forms of muscular dystrophy (MD).

Loss-of-function mutations in SGCA, encoding α-sarcoglycan, cause limb-girdle muscular dystrophy 2D/3R, an early onset, severe and rapidly progressive form of muscular dystrophy affecting equally girls and boys. Patients suffer from muscle degeneration and atrophy affecting the limbs, respiratory muscles, and the heart.

SGCA has 9 coding exons and is expressed in striated muscle (2, 3). Disease-causing mutations are spread along the entire length of the gene without defined mutational hotspots (4, 5). However, some mutations like c.157G>A have been reported more frequently (Fendri K, Kefi M, Hentati F, Amouri R. Genetic heterogeneity within a consanguineous family involving the LGMD 2D and the LGMD 2C genes. Neuromuscul Disord. 2006; 16(5):316-320).

In an embodiment SGCA mutations, in particular c.157G>A mutation, is reversed or repaired by adenine base editing (ABE).

Classical laminopathy refers to diseases caused by mutations in the LMNA gene, coding for the nuclear lamina protein lamin A/C.

In an embodiment LMNA mutations, in particular the c.1366A>G mutation, are reversed or repaired by cytosine base editing (CBE or BE).

Mutations in the DYSF gene, encoding dysferlin, cause weakness mainly of posterior hip and near hip thigh muscles, calf pseudohypertrophy and shoulder muscles. One mutation in DYSF is based on a deletion of G on position 4782 of the DYSF coding sequence, which induces a frameshift and a premature stop codon. Another G>C substitution is four positions downstream.

In an embodiment DYSF mutations, in particular G deletion, are reversed or repaired by CRISPR/Cas9-induced non-homologous end joining (NHEJ). Specifically, a +1A insertion restores the DYSF reading frame resulting in a removal of the premature stop codon.

Calpain 3, the protein encoded by CAPN3, is a cysteine-protease predominantly expressed in skeletal muscle. Mutations in CAPN3 cause LGMD2A, a progressive skeletal muscle disorder without treatment and the most common form of LGMD worldwide. Deletion CAPN3 c.550delA causes a frameshift in exon 4, which creates a premature stop codon.

In an embodiment CAPN3 mutations, in particular A deletion, are reversed or repaired by CRISPR/Cas9-induced non-homologous end joining (NHEJ). Specifically, a +1A insertion restores the reading frame resulting in a removal of the premature stop codon. The +1 insertion in exon 4 of CAPN3 to reverse CAPN3 c.550delA is exempted if the repair is plasmid based. All other approaches such as mRNA- and protein-based repair approaches are included.

As described above, the CRISPR/Cas-based gene editing tools are delivered into the human primary muscle stem cells by at least one DNA- and/or RNA- and/or protein-based carrier, in particular using plasmids, non-integrating viral vectors, recombinant proteins or mRNA as delivery systems. The DNA or RNA delivery system that carries the information for the synthesis of the components required for CRISPR/Cas-based genetic modifications, or the Cas-based proteins, are introduced into primary human stem cells carrying the genetic defect.

The different modes of delivery may thus comprise one or more of the following:
For the Cas enzyme/base editor/prime editor: DNA (e.g. plasmid DNA, minicircle DNA), mRNA, protein, viral vector;
For the single guide RNA: DNA (e.g. plasmid DNA, minicircle DNA), RNA, viral vector;
For the homology template (in case of HDR): DNA (can be single stranded like an ssODN or double stranded like a plasmid of minicircle), viral vector (e.g. AAV)

In one preferred embodiment for delivery, a plasmid is used as transport system. In this case the plasmid may contain DNA encoding for Cas9 and a promoter driven sgRNA expression cassette. For example, an ABE plasmid containing a codon optimized SpCas9 fused to a deaminase domain, T2A-Venus cassette plus human U6 promoter driven sgRNA cassette may be used.

The plasmid containing DNA encoding for Cas-based protein, in particular a base editor, and sgRNA may be transfected into the human primary human stem cell carrying the genetic defect. In one embodiment, transfection was carried out using said human primary muscle cells at a cell density in a range between 40,000 and 90,000 cells/9.5 cm², preferably in a range between 50,000 and 80,000 cells/9.5 cm², such as 55,000 and 75,000 cells/9.5 cm². The required cell density of primary human stem cells is surprisingly low compared for example to cell densities for transfections of induced pluripotent stem cells (iPSC). In case of iPSC transfection, a cell density of about 300,000 cells/9.5 cm² is required.

When using plasmid-based ex vivo delivery a >90% correction of a G>A MD-causing mutation in the gene encoding α-sarcoglycan (SGCA c.157G>A) in primary human MuSC was achieved. The corrected cells exhibited their full myogenic and regenerative potential in xenografts, including reconstitution of the muscle stem cell compartment.

However, plasmid-based delivery carries a risk of transgene integration and leads to a relatively long exposure to gene modifying enzymes.

Thus, in another preferred embodiment mRNA is used as transport system. mRNA-mediated transgene delivery provides transient gene expression whilst eliminating the risk of transgene integration. Transient expression of the gene modifying enzyme reduces the risk of potential off-target mutagenesis, an important aspect for therapeutic development of gene editing strategies.

Nucleofection allows delivery of mRNA to almost 100% of the cells, resulting in highly uniform transgene expression levels and eliminating the need for reporter genes or enrichment steps to obtain a homogenous population of gene edited primary muscle stem cells.

In an embodiment, transfection of mRNA to almost 100% of MuSC was achieved by using nucleofection.

As mentioned above in yet a further preferred embodiment, a Cas-based recombinant protein is used as mode of delivery for CRISPR/Cas-based gene editing tools.

When delivering recombinant proteins, no synthesis is required in the cell because the proteins are already produced in vitro (usually in bacteria) and introduced into the cells. The Cas-based protein (like ABE8e in the example provided below) is usually pre-incubated with the sgRNA or crRNA:tracrRNA heteroduplex so that the so-called ribonucleoprotein (RNP) complex is formed. Subsequently, the RNPs are delivered to the cell. However, both components can also be delivered to the cells separately and they will form a complex inside the cell. Nucleofection is applied for protein mediated gene editing.

In one embodiment, ribonucleoprotein (RNP) complexes comprised of ABE8e recombinant protein and an sgRNA designed to mutate the splice donor site of NCAM1 exon 7 were assembled. ABE8e protein and the respective sgRNA were mixed to form the RNP complexes.

The RNP complex is subsequently transfected into the muscular human stem cells by using nucleofection.

As mentioned, the method for providing a sample of an isolated muscle-fiber containing tissue sample collected from at least one patient with muscular dystrophy has been previously described (WO 2016030371 A1).

Specifically, the primary stem cells from said muscle-fiber containing tissue sample collected from a patient with muscular dystrophy are cultivated by a treatment without oxygenation under hypothermic conditions having a defined temperature and a defined atmosphere, wherein the temperature does not exceed 15° C. and the atmosphere has an oxygen content not exceeding 21% (v/v), for a time of 4 days to 4 weeks.

The cultivation under these conditions leads to an enrichment of stem cells in the sample such that approximately 70 to 100% of all viable cells in the sample are cultivated stem or derivatives from cultivated stem cells after a first period of time. The cultivation takes place by using a medium that is suited or adapted for the stem cells to be cultivated.

In an embodiment, the temperature does not exceed 14° C., in particular 13° C., in particular 12° C., in particular 11° C., in particular 10° C., in particular 9° C., in particular 8° C., in particular 7° C., in particular 6° C., in particular 5° C., in particular 4° C., in particular 3° C., in particular 2° C. in particular 1° C., in particular 0° C. In an embodiment, the temperature is in a range of 0° C. to 15° C., in particular 1° C. to 14° C., in particular 2° C. to 13° C., in particular 3° C. to 12° C., in particular 4° C. to 11° C., in particular 5° C. to 10° C., in particular 6° C. to 9° C., in particular 7° C. to 8° C.

In an embodiment, the atmosphere has an oxygen content not exceeding 20 volume %, in particular 19% (v/v), in particular 18% (v/v), in particular 17% (v/v), in particular 16% (v/v), in particular 15% (v/v), in particular 14% (v/v), in particular 13% (v/v), in particular 12% (v/v), in particular 11% (v/v), in particular 10% (v/v), in particular 9% (v/v), in particular 8% (v/v), in particular 7% (v/v), in particular 6% (v/v), in particular 5% (v/v), in particular 4% (v/v), in particular 3% (v/v), in particular 2% (v/v), in particular 1% (v/v), in particular not exceeding any of the before-mentioned oxygen contents. Conditions having an atmosphere with an oxygen content of less than 20% (v/v) are often also referred to as hypoxic conditions.

In an embodiment, the atmosphere has an oxygen content lying in a range of 1% (v/v) to 21% (v/v), in particular of 2% (v/v) to 20% (v/v), in particular of 3% (v/v) to 19% (v/v), in particular of 4% (v/v) to 18% (v/v), in particular of 5% (v/v) to 17% (v/v), in particular of 6% (v/v) to 16% (v/v), in particular of 7% (v/v) to 15% (v/v), in particular of 8% (v/v) to 14% (v/v), in particular of 9% (v/v) to 13% (v/v), in particular of 10% (v/v) to 12% (v/v), in particular of 3% (v/v) to 11% (v/v).

In an alternative embodiment, the atmosphere has an oxygen content not exceeding 30 volume %, in particular not exceeding 29% (v/v), in particular not exceeding 28% (v/v), in particular not exceeding 27% (v/v), in particular not exceeding 26% (v/v), in particular not exceeding 25% (v/v), in particular not exceeding 24% (v/v), in particular not exceeding 23% (v/v), in particular not exceeding 22% (v/v), in particular not exceeding 21% (v/v),

In an embodiment, the temperature is in a range of 0° C. to 10° C. and the oxygen content is in a range of 0% (v/v) to 8% (v/v). In an embodiment, the temperature is in a range of 2° C. to 5° C. and the oxygen content is in a range of 2% (v/v) to 5% (v/v). In an embodiment, the temperature is in a range of 3° C. to 4° C. and the oxygen content is in a range of 3% (v/v) to 4% (v/v). In an embodiment, the temperature does not exceed 10° C. and the oxygen content does not exceed 8% (v/v).

In an embodiment, growth factors are added to the medium in which the stem cells are cultivated. In an embodiment, growth factors are only added if the stem cells are cultivated for more than 2 weeks, in particular for more than 2 weeks at 4° C. In particular in case of HMFF as sample and satellite cells to be cultivated, a medium with low serum content is suited. A well-suited medium is a serum-reduced optimized minimal essential medium, such as OptiMEM, obtainable from Life Technologies. It turned out that a duration of the first period of time of 5 days to 2 weeks, in particular of 6 days to 1 week, in particular 1 week is particularly suited for stem cell cultivation and enrichment.

In an embodiment, the sample is an isolated tissue sample. It can be isolated from a patient by standard methods, such as a biopsy. In an embodiment, the sample is an isolated muscle fiber fragment. Human muscle fiber fragments (HMFFs) are particularly suited and easily obtainable from a muscle biopsy specimen.

In an embodiment, the stem cells are cultivated in a united cell structure. This can be achieved best by a supportive structure that enables a united cell structure. In an embodiment, this supportive structure is the natural structure in which the cells grow in a body (such as a HMFF which is very well suited in the context of this solution to cultivate satellite cells). In another embodiment, this supportive structure is an artificial structure mimicking or closely resembling the natural structure in which the cells grow in a body.

After a sufficient amount of primary stem cells are cultivated the above described gene editing methods using CRISPR/Cas-based tools are applied to the primary stem cells in order to correct the mutations in the muscle genes.

Following gene repair, the human muscle cells are screened for the desired gene repair/editing event. A selection step, for example by FACS, may be performed. In particular, a fluorescent molecule expressed by transfected/edited cells may be used, or selection may be based on live cell staining of extracellular epitopes.

The genetically modified primary stem cells are further cultivated.

In one embodiment the modified primary stem cells are fused into multinucleated myotubes in vitro.

In a further aspect, the solution also relates to genetically repaired or modified human muscle stem cells obtained after carrying out above method.

In an embodiment the genetically repaired human muscle stem cells comprise at least one gene encoding for at least one muscle protein, wherein the at least one gene underwent a targeted modification of at least one mutation in said gene, for example by NHEJ, base editing or prime editing using CRISPR/Cas-based tools as described in detail above.

As previously indicated, a genetically corrected muscle stem cell obtained by the present method differs from healthy (“wild-type”) muscle stem cells, and is thus not identical to the wild-type. This has been demonstrated in base-edited human fibroblasts carrying a mutation in LMNA (Koblan et al., In vivo base editing rescues Hutchinson-Gilford progeria in mice. Nature 2021; doi.org/10.1038/s41586-020-03086-7, FIG. 2d). It has to be emphasized that “wild-type” MuSC from muscular dystrophy patients do not exist naturally.

In particular, site specific insertions and deletions cause frameshifts. Such reframing often results in a DNA sequence different than wild-type. Site specific insertion may restore a reading frame but the protein encoded by such a restored reading frame may differ from the wild type as shown for DYSF or CAPN3 gene (see Examples further below),

In a preferred embodiment, the genetically repaired human muscle stem cells comprise at least one modified gene encoding for at least one of the following muscle proteins: LMNA encoding for lamin A/C, CAPN3 encoding for calpain 3, DYSF encoding for dysferlin, SGCA encoding for α-sarcoglycan, VCP encoding for valosin containing protein, PAX7 encoding for paired box 7, NCAM1 encoding for neural cell adhesion molecule 1, DMD encoding for dystrophin.

In a further aspect, the solution also relates to the medical use of genetically repaired or modified muscle stem cells. Thereby, the stem cells are intended to be used in cell replacement therapies for muscular dystrophy (MD), including all types of limb-girdle muscular dystrophy (LGMD), in particular of type LGMD1/D, LGMD2/R (Straub et al., 2018; https://doi.org/10.1016/j.nmd.2018.05.007), all X-linked muscular dystrophies, in particular Emery-Dreyfuss MD, Duchenne MD, Becker MD, and all MDs caused by repeat expansion (i.e. myotonic dystrophy type 1 and type 2) or repeat deletion (facioscapulohumeral muscular dystrophy) mutations. The stem cells can be also used for treatment of Pax7 myopathy (Marg et al., Nat Commun. 2019 Dec. 18; 10(1):5776. doi: 10.1038/s41467-019-13650-z) or VCP myopathy.

Herewith, a method of transplanting cultivated stem cells to a subject or patient in need thereof is disclosed, the method comprising administering genetically repaired or modified stem cells cultivated as outlined above to the patient, in particular by autologous transplantation.

Herewith, a method of treating a patient suffering from a muscular dystrophy is disclosed, the method comprising administering genetically modified muscular stem cells to the patient. In an embodiment, the muscular stem cells are satellite cells. In an embodiment, the genetically modified stem cells are administered as cell suspensions. Delivery in form of a tissue supportive structure system is also feasible.

### BRIEF DESCRIPTION OF THE DRAWINGS

The solution will be explained in more detail in the following with respect to exemplary embodiments and Figures.

FIGS. 1A-E show ABE repairs the SGCA c.157G>A mutation in patient and carrier primary muscle stem cells without detectable off-target editing.

FIG. 2 shows CBE repairs the LMNA c.1366 A>G mutation in patient-derived cells.

FIG. 3 shows DYSF editing in patient primary MuSC/myoblasts.

FIG. 4 shows mRNA-mediated ABE7.10 delivery leads to almost 100% disruption of a NCAM1 splice donor in human primary MuSC.

FIG. 5 shows mRNA-mediated delivery of ABE7.10 repairs the SGCA c.157G>A mutation in human primary MuSC.

FIG. 6 shows Optimization of GFP mRNA nucleofection in human primary MuSC: Transfection efficiency and cell viability with different nucleofection programs.

FIG. 7 shows Comparison of ABE7.10 mRNA-mediated repair efficiency of the SGCA c. 157G>A mutation in human primary MuSC with two different nucleofection programs.

FIG. 8 shows ABE8e protein-mediated disruption of an NCAM1 splice donor in human primary MuSC.

### DETAILED DESCRIPTION

### Example 1: Gene Editing on SGCA Related Muscular Dystrophy Patient-Derived Cells

a) Isolation of primary MuSC from a patient and a carrier with a compound heterozygous SGCA c.157G>A mutation.

Primary MuSC from muscle biopsy specimens obtained from a 10-year old male LGMD2D patient carrying a compound heterozygous SGCA c.157G>A mutation and from a related carrier were isolated and characterized.

Primary MuSC isolation and culture. Immediately after the biopsy procedure, the muscle specimen was transferred into Solution A for transport (30 mM HEPES, 130 mM NaCl, 3 mM KCl, 10 mM D-glucose and 3.2 μM Phenol red, pH 7.6). The fresh muscle specimen was manually dissected, and fragments were subjected to hypothermic treatment at 4-6° C. for 2 to 7 days prior to downstream processing for MuSC isolation. Oligoclonal MuSC colonies were obtained following mechanical dissection as described (Marg A, et al. Human muscle-derived CLEC14A-positive cells regenerate muscle independent of PAX7. Nat Commun. 2019; 10(1):5776). The outgrowing colonies were expanded until passage 4 and characterized prior to cryopreservation. To enhance the probability of available MuSC in difficult-to-handle biopsy specimens, classical purification was performed in parallel (Blau H M, Webster C, Pavlath G K. Defective myoblasts identified in Duchenne muscular dystrophy. Proc Natl Acad Sci USA. 1983; 80(15):4856-4860). All cell populations used in this study were >95% positive for Desmin. To induce myoblast-to-myotube fusion, medium was switched to Opti-MEM I Reduced Serum Media (Thermo Fisher Scientific) once cells reached confluence.

Primary MuSC cultures from patient and carrier were 95-100% Desmin+ and expressed the myogenic markers Pax7, MyoD, Myf5 and the proliferation marker Ki-67. The c.157G>A mutation affects the last coding nucleotide of exon 2.

b) ABE results in >90% correction of SGCA c.157G>A in primary human MuSC without detectable off-target editing

It was found that c.157G>A is an ideal ABE target, as it is located 15 bp upstream of an -NGG PAM (equivalent to protospacer position 6, thus in the center of the ABE activity window). No other adenines are located within the ABE activity window, so undesired bystander edits are unlikely. It was first assessed if ABE can be used to repair the c.157G>A mutation in patient iPSC.

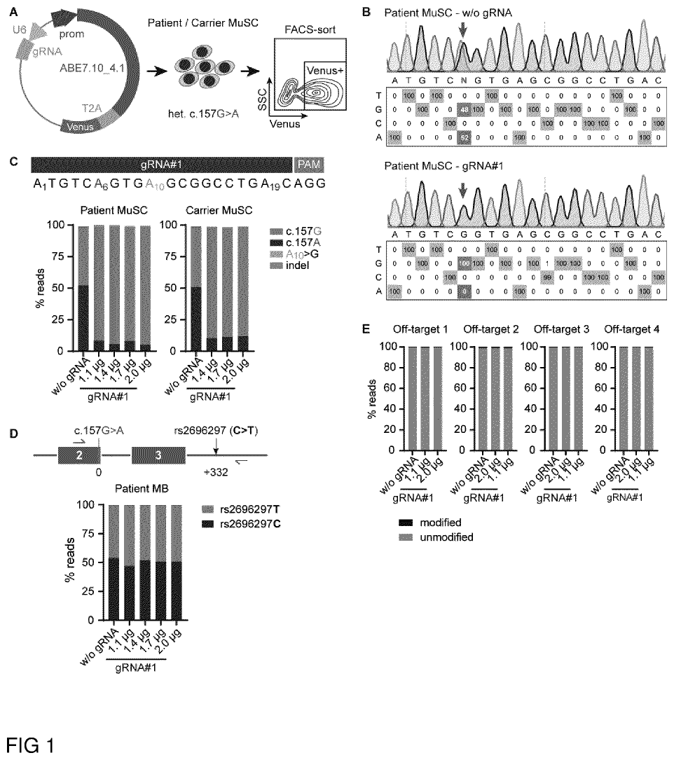

The MuSC from the patient were transfected with various amounts of a plasmid encoding ABE7.10_4.1 (FIG. 1A) a vector based on ABE7.10 (Gaudelli N M, et al. Programmable base editing of A⋅T to G⋅C in genomic DNA without DNA cleavage. Nature. 2017; 551(7681):464-471) containing a codon-optimized Cas9(D10A) nickase N-terminally fused to the TadA heterodimer, followed by a T2A-Venus cassette under control of the CAG promoter, and a U6 promoter-driven sgRNA expression cassette.

It was enriched for Venus-positive cells (FIG. 1A) via FACS and ABE efficiency was assessed using EditR (Kluesner M G, et al. EditR: A Method to Quantify Base Editing from Sanger Sequencing. CRISPR J. 2018; 1(3):239-250).

Human primary MuSC transfection and sorting. Human primary MuSC were plated one day before transfection at a density of 55,000 cells/9.5 cm² in Skeletal Muscle Cell Growth Medium (SMCGM, Provitro) and transfected using Lipofectamine@3000 (Thermo Fisher Scientific) following manufacturer's instructions. SMCGM was exchanged after one day. Two days after transfection, cells were collected for FACS-sorting in PBS containing 50% SMCGM, 0.05 mM EDTA and 100 μg/ml Primocin^{T}. Venus-positive cells were sorted using a FACSAria Fusion cell sorter (BD Biosciences) and cultured in SMCGM. 100 μg/ml Primocin™ were added to the culture medium for two days.

All vector concentrations resulted in >99% c.157G nucleotide rates in patient and carrier MuSC as analyzed by EditR (FIG. 1B). Then amplicon sequencing was performed with subsequent analysis by Crispresso2 (26) and confirmed high c.157G nucleotide rates of >90% for patient MuSC and >85% for carrier MuSC (FIG. 1C). Bystander A>G editing at protospacer position 10 was detected in a very low (0.2-2%) percentage of reads. In two samples 1.1% and 0.3% of reads containing indels were detected. Omission of gRNA did not result in either A>G editing or indels (FIG. 1C).

An equal representation of both alleles in the amplicon sequencing data was confirmed, thus ruling out detection bias (FIG. 1D).

No Cas9-dependent off-target editing events were detected at the top predicted (by CRISPOR) off-target loci containing A nucleotides in the ABE activity window with either the lowest or highest vector concentration (FIG. 1E).

It was found that ABE7.10_4.1 induced efficient c.157A>G conversion when combined with a suitable gRNA (gRNA #1), with only minimal (0.2-2%) bystander A>G edits detected by amplicon sequencing and Crispresso 2 analysis.

It was thus concluded that the SGCA c.157G>A mutation can be repaired in human primary MuSC with very high efficiency and specificity via ABE. Repaired SGCA c.157G>A is hereafter referred to as SGCA c.157Grep.

c) SGCA c.157Grep primary MuSC show normal α-sarcoglycan mRNA and protein expression.

To assess the functional outcome of ABE, α-sarcoglycan mRNA and protein expression in SGCA c.157Grep myotubes was analyzed. It was found that the splicing defect was rescued as shown by the increase in α-sarcoglycan transcripts containing exon 2 in SGCA c.157Grep compared to unedited patient and carrier myotubes, reaching levels similar to control 3 (het. c.748-2A>G carrier) in the case of patient myotubes. Furthermore, total SGCA mRNA levels increased in patient myotubes following ABE, probably because co-skipping of exons 2+3 (but not exon 2 alone) induces a frameshift leading to a premature stop codon and thus likely nonsense mediated mRNA decay (NMD). Western blot and immunostaining analysis revealed that α-sarcoglycan protein was restored in SGCA c.157Grep patient cells.

d) SGCA c.157Grep primary patient MuSC are viable, proliferative, and myogenic.

Primary cells are especially susceptible to stress induced by extensive manipulation. Primary MuSC derived from MD patients with mutations in genes responsible for membrane integrity are particularly vulnerable. A decrease in cell proliferation in the first days following transfection and sorting as compared to untransfected patient MuSC was observed. However, Venus-positive cells (>48% of the source cell population) proliferated extensively after sorting and were further expanded for at least 2-3 passages before cryopreservation. SGCA c.157Grep primary MuSC could readily fuse into multinucleated myotubes in vitro. Moreover, the pattern of α-sarcoglycan localization was indistinguishable from control myotubes.

e) SGCA c.157Grep primary MuSC regenerate muscle and repopulate the satellite cell niche in vivo.

SGCA c.157Grep primary MuSC were transplanted into irradiated anterior tibial muscles of immunocompromised NSG mice. It was found that SGCA c.157Grep patient MuSC gave rise to abundant human muscle fibers. Furthermore, the satellite cell niche between the sarcolemma and the basal lamina was populated with numerous Pax7+ cells of human origin. Taken together, SGCA c.157Grep patient MuSC are capable of both myofiber regeneration and reconstitution of the satellite cell compartment in vivo.

Human MuSC transplantation. SGCA c.157Grep patient MuSC that were 99% Desmin+, 27% Pax7+, 25% Ki-67+, 66% MyoD+ and 40% Myf5+ were used for transplantation. 6-week old male NOD.Cg-Prkdc^{scid}Il2rg^{tm1Wjl}/SzJ (NSG) mice were purchased from Charles River Laboratories 1 week before the experiment. Animal housing and hygienic monitoring followed FELASA recommendations. Focal irradiation of the recipient hind limbs was performed two days prior to cell transplantation as described (17, 18). Two injections of 5.5 μl containing 2,5×10⁴ cells in a sterile PBS+2% FCS solution were performed following parallel trajectories into the medial portion of the TA muscle (in total 5×10⁴ cells per grafted muscle) as described (18). Mice were sacrificed 19 days after cell transplantation. TA muscles were cryopreserved in liquid nitrogen-chilled isopentane, mounted in gum tragacanth and stored at −80° C.

### Example 2: Gene Editing in LMNA Related Muscular Dystrophy Patient-Derived Cells

Classical laminopathy refers to diseases caused by mutations in gene LMNA, coding for nuclear lamina protein lamin A/C. The state-of-the-art gene editing tools provide the possibility to correct the mutations at the genomic level, especially the powerful base editors in correcting single nucleotide mutations without DNA double strand breaks.

An 8-year-old girl was diagnosed with muscular dystrophy, carrying a dominant mutation in LMNA c.1366 A>G. Base editing was performed at first with patient derived induced pluripotent stem cells (iPSC), an unlimited cell source to test the editing efficiencies.

The initial test was done with co-transfection of two vectors to iPSC with transfection reagent Lipofectamine®3000 in mTeSR Plus stem cell medium. One DNA vector carries CBE4max—an enhanced version of the first reported cytidine base editor and SpRY, a new near-PAMless Cas9 able to edit mutations that were previously uneditable by classical Cas9, and the other vector expresses the sgRNA.

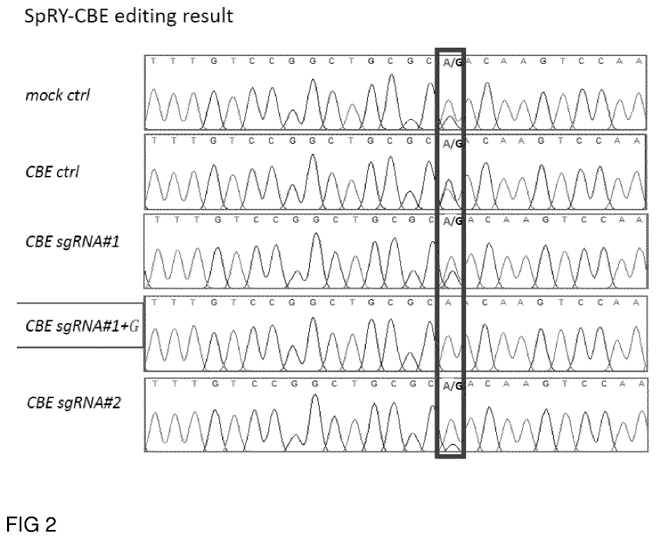

Initial editing results showed a conversion of G to A, although the editing efficiency is low due to low transfection efficiency (FIG. 2).

Based on the preliminary results of an ABE editing project, the transfection with synthesized Cas9 mRNA instead of a DNA vector revealed higher editing efficiency. Thus editing efficiency can be improved via transfection with the synthesized mRNA containing the CBE4max and SpRY sequences along with synthesized sgRNA.

Following by the optimized CBE editing in iPSC, patient derived muscle stem cells will be edited with the same protocol and ultimately the corrected muscle stem cells will be used for the transplantation therapy to improve the muscle function of patients.

### Example 3: DYSF Gene Editing in Patient Primary MuSC/Myoblasts

a) Human Primary MuSC Culture

Human MuSC were grown in humidified atmosphere containing 5% CO₂ at 37° C. on 10 cm plastic dishes (Corning) in skeletal muscle cell growth medium (SMCGM) (Provitro) enriched with fetal calf serum (FCS) supplement mix (Provitro) and 2.72 mM glutamine (GlutaMAX^{T}M, Thermo Fisher Scientific). For cell passaging, human MuSC were washed with Dulbecco's phosphate-buffered saline (DPBS) (Thermo Fisher Scientific) and treated with 0,25% Trypsin/EDTA (Thermo Fisher Scientific) at 37° C. for 5 min.

Detached cells were collected in SMCGM+supplement to a dilution of 1:10 and centrifuged at 200 g for 5 min at room temperature (RT). Pellet was resuspended in an appropriate volume of SMCGM+supplement and seeded at a density of 1-2*10⁴ cells/cm² on 10 cm plates. Cells were passaged every 2-3 days according to growth rate/confluence.

b) CRISPR/Cas9-Based Gene Editing

TABLE 1
sgRNA sequences used for the
CRISPR/Cas9 experiment.

| | Target | Guide | Guide | | |
|---|---|---|---|---|---|
| Locus | allele | ID | sequence | PAM | Orientation |
| DYSF | Mutant | DYSFex44 | AAATAGGGG | GGG | sense |
| | exon 44 | mut#3 | TCCAGCGTG | | |

c) Lipo-Transfection of the Cas9/sgRNA Complex

For the CRISPR/Cas9 experiments, human MuSC were seeded at a density of 75,000 cells/well of a 6-well plate one day before transfection. 1 μg SpCas9::Venus plasmid DNA (with and without sgRNA) was transfected using Lipofectamine@3000 transfection reagent (Invitrogen, Germany), according to the manufacturer's instructions. 48h after transfection the Venus-positive cells were sorted using FACSAria Fusion (BD). Sorted cells were plated again and expanded for genomic DNA isolation and dysferlin protein analysis via flow cytometry or immunofluorescence staining, respectively.

For dysferlin immunostaining, human MuSC were seeded in 8-well ibidi μ-Slides (IBIDI GmbH Martinsried, Cat. #80826) in SMCGM+supplement and allowed to proliferate until 70%/80% confluence was reached. Myoblast fusion was induced by switching the culture medium to OptiMEM (Thermo Fisher Scientific). After four days, cells were fixed and stained with an antibody against the N-terminal part of Dysferlin (ab124684, Abcam).

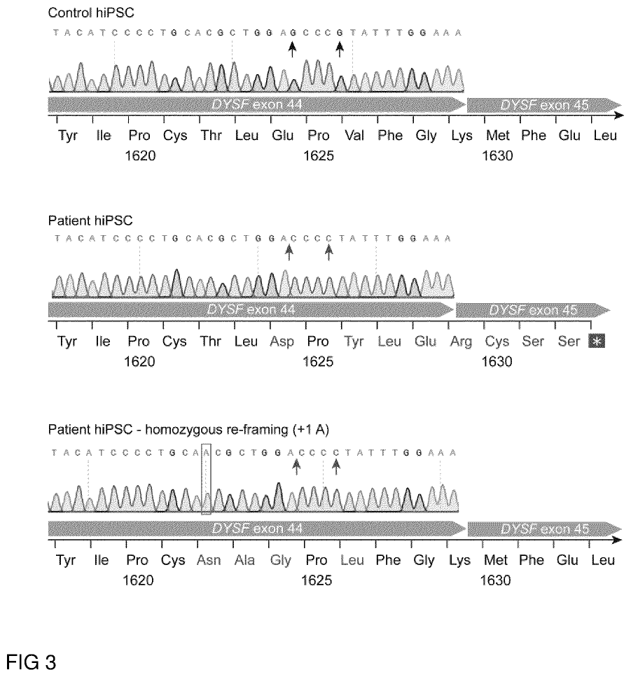

FIG. 3 illustrates the results obtained for DYSF gene editing. Genomic DNA was isolated from a control (upper panel) and a patient carrying a homozygous DYSF c.4782_4786delinsCCC mutation in exon 44 (middle panel). The c.4782_4786delinsCCC mutation is a deletion of a G nucleotide in position c.4782 of the DYSF coding sequence (which induces a frameshift) and a G>C substitution four positions downstream.

The +1A insertion restores the DYSF reading frame, resulting in a removal of the premature Stop codon, whilst four amino acids (indicated in blue) differ from the wild-type protein sequence (lower panel). Dysferlin protein expression is rescued in patient MuSC and derived myotubes after restoring the reading frame by the +1A insertion. Dysferlin localization in +1A re-framed patient myotubes is similar to control myotubes.

### Example 4: CAPN3 Gene Editing in Patient Primary Myoblasts

T

Calpain 3, the protein encoded by CAPN3, is a cysteine-protease predominantly expressed in skeletal muscle. Mutations in CAPN3 cause limb-girdle MD Type 2A (LGMD2A), a progressive skeletal muscle disorder without treatment and the most common form of LGMD worldwide.

Human primary muscle stem cells from 35 patients carrying 37 different CAPN3 mutations were isolated and expanded. 20% of the patients carry the well-known founder mutation CAPN3 c.550delA causing a frame shift in exon 4, which creates a premature stop codon. In most cases, patients carry compound heterozygous CAPN3 c.550delA mutations, two patients with homozygous c.550delA mutations are also part of the cohort.

Primary MuSC from a homozygous patient were isolated, expanded and transfected with a plasmid, which carries mutation-specific sgRNAs and SpCas9::Venus. After cell sorting and expansion, a subsequent in-depth sequence analysis of the CAPN3 c.550 DNA region showed base insertions and deletions (indels) at the targeted CAPN3 locus with an efficiency of up to 60%. One of the sgRNAs had a preference of a +1 bp insertion at the position of the mutation demonstrating an indel signature bias of specific sgRNAs and reframing of the open reading frame (ORF).

The effects on protein level were analyzed using a custom made monoclonal anti-Calpain 3 antibody suitable for immunostaining.

### Example 5: mRNA-Mediated Delivery of Cas9 Gene Editing Tools to Human Primary Muscle Stem Cells

a) mRNA Nucleofection Results in Close to 100% Transfection Efficiency of Primary Human MuSC with Minimal Toxicity

Primary MuSC were harvested using TrypLE Express, spun down for 5 minutes at 200 g, and washed once with DPBS. After a second spin down, the supernatant was removed and the cells were resuspended in P5 Primary Cell Nucleofector Solution (Lonza, Basel, Switzerland) already premixed with mRNA (SpCas9 mRNA, Aldevron, ND, USA; ABE7.10 mRNA, AmpTec GmbH, Hamburg, Germany; GFP mRNA, Aldevron) or sgRNA (IDT/Synthego, CA, USA) at a concentration of 7.5×10⁶ cells per ml. For 3 μg of gene editing molecule-encoding mRNA, 2 μg of 5′/3′ end-modified sgRNA (1:0.67 ratio) were added to a 20 μl reaction. The reaction can be scaled up linearly. The cells were electroporated with the Amaxa 4D Nucleofector (Lonza, Basel, Switzerland) using the X Unit with 16-well nucleofection cuvettes. After nucleofecting the cells, 80 μl of prewarmed SMCGM was added and cells were transferred to a single well of a 6-well plate containing 2 ml of prewarmed SMCGM. The cell culture medium was changed the day after.

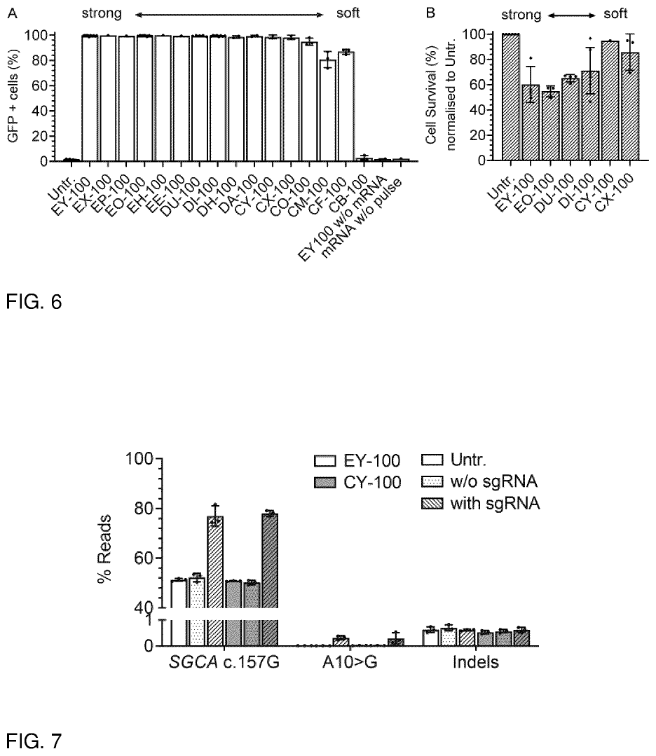

Cell fitness and viability are crucial if edited cells are intended for use in transplantation therapies. To determine parameters resulting in high transfection efficiency, measured by GFP+ cells, and minimal cellular toxicity, we compared different nucleofection programs. Nucleofection was optimized by reducing gradually pulse (namely, nucleofection program) intensitiesThe pulse codes EY-100, EX-100, EP-100, EO-100, EH-100, EE-100, DU-00, DI-100 DH-100, DA-100, CY-100, CX-100, and CO-100 led to transfection efficiencies of >=95% (FIG. 6A). The cell viability after nucleofection gradually increased from the strongest pulse code, EY-100, to the softer pulse codes CY-100, and CX-100 (FIG. 6B). To confirm that editing efficiencies remained identical after pulse code optimization, we compared editing outcomes using strong (EY-100) and soft pulse (CY-100) parameters (FIG. 7). No significant differences could be observed.

b) mRNA-Mediated Delivery of SpCas9 Results in Highly Efficient Gene Editing in MuSC from Many Donors

To develop and systematically assess a pipeline for mRNA-mediated delivery of gene editing tools to primary human MuSC, a universal read-out system relevant to MuSC from all donors was established. For that purpose, a strategy to target the gene encoding neural cell adhesion molecule 1 (NCAM1), a membrane protein expressed by all human MuSC and myoblasts with an extracellular domain easy to detect in living cells was designed. mRNA encoding S. pyogenes Cas9 (SpCas9) to MuSC from six donors of different ages and genders were delivered. Transfection of a range of SpCas9 mRNA concentrations and an sgRNA targeting NCAM1 exon 3 at a constant ratio resulted in efficient indel formation, with the highest editing rate observed for 2 μg of SpCas9 mRNA. Gene editing led to comparable rates of NCAM1 protein knock-out as assessed by immunofluorescence staining and flow cytometry. An increase in the percentage of edited MuSC from day 2 to day 8 after nucleofection was observed for all donors, reaching indel rates of up to >90% at day 8. Consistently, NCAM1-positive cells decreased between day 4 and 6 after nucleofection and remained constant thereafter.

c) mRNA-Mediated Delivery of ABE7.10 Results in Highly Efficient Selection-Free Base Editing of Human MuSC

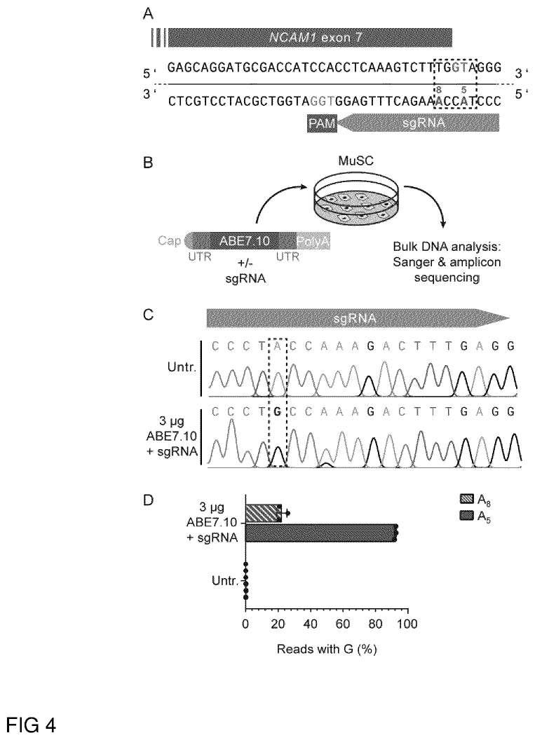

mRNA-based delivery of base editor ABE7.10 was investigated. An sgRNA was designed to mutate the splice donor site of NCAM1 exon 7 (FIG. 4A). mRNA-delivery of ABE7.10 and the corresponding sgRNA resulted in >90% A to G conversion of the target adenine located in the center of the ABE activity window, at protospacer position 5 (A₅), as confirmed via amplicon sequencing (FIGS. 4B to 4D). A neighboring adenine at protospacer position 8 (A₈) was co-edited in up to 22% of sequencing reads. In contrast, plasmid-based experiments using the identical splice donor targeting strategy resulted in a lower mean editing efficiency and lower rates of A₅ versus A₈ to G conversion (not shown). The described nucleotide changes at A₅ and A₈ did not result in a clear NCAM1 protein knock-out.

d) Human MuSC Retain their Myogenic and Proliferative Properties Following mRNA-Mediated Gene and Base Editing

To determine if mRNA-mediated delivery of gene editing tools or knock-out of NCAM1 altered the myogenic or proliferative properties of human MuSC, the expression profiles of myogenic and proliferation markers of passage-matched unedited and edited cells from the same donor were analyzed. Purity of the MuSC populations remained constant and higher than 95% as determined by the myogenic marker Desmin (DES) and counterstained with the fibroblast marker TE7. The myogenic transcription factors PAX7, MYF5, and the proliferation marker KI-67 were similarly expressed in edited and unedited MuSC. KI-67-positive, proliferating MuSC varied between cell populations from 30% to 60% before editing and after editing (not shown). Next the differentiation capacity of edited MuSC in vitro with myoblast fusion assays was assessed. All edited and unedited MuSC populations gave rise to multinucleated myotubes with the typical striated pattern (not shown). Fusion indices remained constant between donor- and passage-matched untransfected cells, and cells edited by SpCas9 or ABE7.10 mRNA and the respective sgRNA (not shown).

e) mRNA-Based ABE Delivery Efficiently Corrects the SGCA c.157G>a Muscular Dystrophy-Causing Mutation in Human MuSC

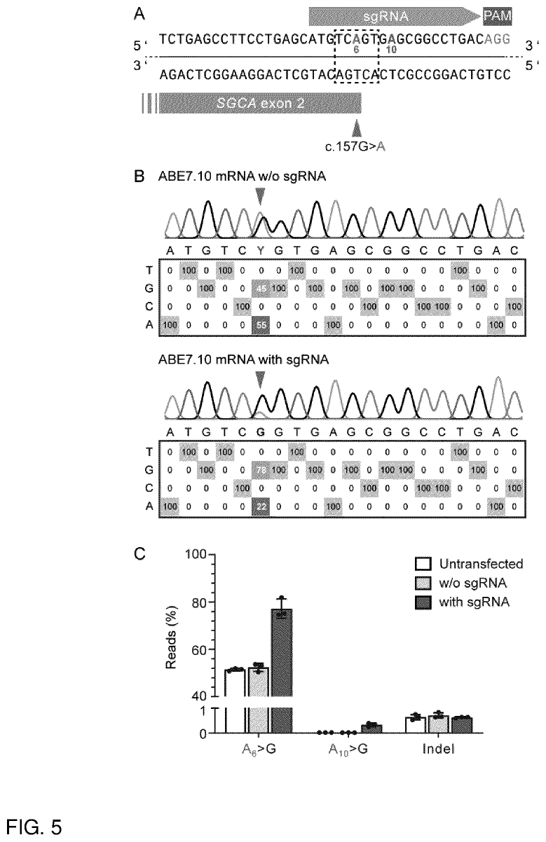

Human MuSC carrying a heterozygous SGCA c.157G>A mutation were transfected with ABE7.10 mRNA and the corresponding sgRNA (FIG. 5A). mRNA-mediated base editing efficiently repaired the mutation, resulting in c.157G nucleotide rates of >80%. Bystander editing of the adenine located at protospacer position 10 was found in a very low percentage of reads<0.4% and no indels specific for mRNA-transfected or edited samples were detected (FIG. 5B, 5C).

### Example 6. Nucleofection of ABE8e Recombinant Protein and an sgRNA Results in Disruption of the NCAM1 Exon 7 Splice Donor Site in Human Primary MuSC

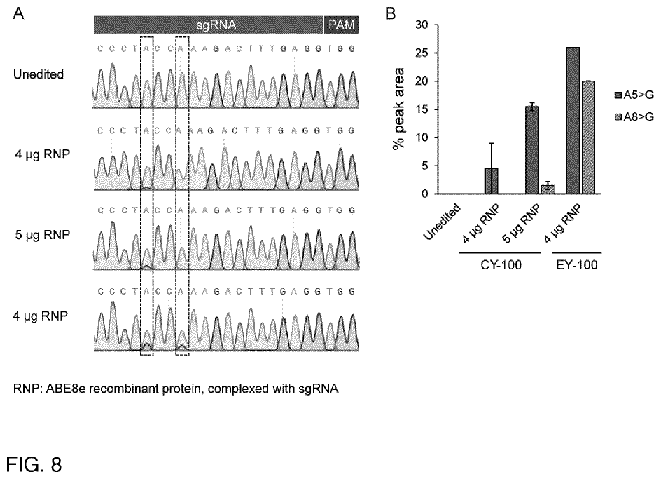

First, ribonucleoprotein (RNP) complexes comprised of ABE8e recombinant protein and an sgRNA designed to mutate the splice donor site of NCAM1 exon 7 were assembled (ABE8e recombinant protein was produced by the Max-Delbruck Center protein core facility and sgRNA was purchased from Integrated DNA Technologies, IDT). The target adenine conforming the consensus NCAM1 exon 7 splice donor site is located in position 5 of the protospacer. An additional adenine is present within the ABE editing window, in protospacer position 8 (FIG. 8—target adenine: A₅; bystander edit: A₈). ABE8e protein and the respective sgRNA were mixed gently in sterile PBS at a molar ratio of 1:1.2 (ABE8e:sgRNA) and the mix was incubated for 20 minutes to 1 hour at room temperature to form the RNP complexes. The RNP were then stored at 4° C. until nucleofection. The final concentration of ABE8e protein in the RNPs was 2 μg/μl. Cultured primary MuSC were harvested using TrypLE Express and centrifuged for 5 minutes at 200 g. They were resuspended in PBS and counted. A suitable number of cells was centrifuged again at 200 g. The supernatant was aspirated and the cell pellet was resuspended in P5 Primary Cell Nucleofector Solution (Lonza, Basel, Switzerland) containing the RNPs. 150,000 cells were nucleofected in a 20 μl reaction using an Amaxa 4D Nucleofector, in particular the X Unit with 16-well nucleofection cuvettes (Lonza, Basel, Switzerland), using the pulse codes CY-100 or EY-100. After nucleofection, 80 μl of prewarmed Skeletal muscle cell growth medium+Supplement (SMCGM, Provitro) were added to each cuvette to harvest the cells, and they were transferred to either one (for MuSC nucleofected with the pulse code CY-100) or two wells (for MuSC nucleofected with the pulse code EY-100) of a 6-well plate containing 2 ml of prewarmed SMCGM. Medium was exchanged every other day and cells were expanded and subsequently harvested for genomic DNA isolation 7 days after nucleofection. Base editing was analyzed by PCR amplification of the target locus followed by Sanger sequencing. The % peak area corresponding to A or G was calculated using the online tool EditR (Kluesner M, Nedveck D, Lahr W, Moriarity B. EditR: A method to quantify base editing via Sanger sequencing. The CRISPR Journal. 2018). Edited MuSC preserved their myogenic and proliferative properties based on microscopic determination of the percentage of cells stained positive for the myogenic markers Desmin, PAX7, MYF5, MYOD, and the proliferation marker KI-67, and negative for the fibroblast marker TE7 (not shown).

1. An ex vivo method for gene repair in primary human muscle stem cells (satellite cells)
comprising the following steps
providing a sample of an isolated muscle-fiber containing tissue sample collected from at least one patient with monogenic muscle disease, wherein the monogenic muscle disease is caused by at least one mutation in at least one gene encoding for at least one muscle protein;
isolating and cultivating primary stem cells from said muscle-fiber containing tissue sample, and
correcting the at least one mutation in the at least one gene encoding for at least one muscle protein in the cultivated primary stem cells by targeted modification of the at least one mutation by gene editing using CRISPR/Cas-based tools,
wherein the CRISPR/Cas-based gene editing tools are delivered to the cultivated primary muscle stem cells by at least one DNA- and/or RNA- and/or protein-based carrier, in particular plasmids, non-integrating viral vectors, mRNA or protein.

2. The method according to claim 1, herein the monogenic muscles disease comprises one of the following: muscular dystrophy including all types of limb-girdle muscular dystrophy (LGMD), in particular of type LGMD1/D, LGMD2/R, all X-linked muscular dystrophies (Emery-Dreyfuss MD, Duchenne MD, Becker MD), all MDs caused by repeat expansion (i.e. myotonic dystrophy type 1 and type 2) or repeat deletion (facioscapulohumeral muscular dystrophy) mutations, Pax7 myopathy or VCP myopathy.

3. The method according to claim 1, wherein the at least one gene mutation can be a deletion, insertion or point mutation, repeat expansion, or repeat deletion, in particular a deletion or point mutation.

4. The method according to claim 1, wherein the at least one mutation is located in at least one of the following genes: LMNA encoding for lamin A/C, CAPN3 encoding for calpain 3, DYSF encoding for dysferlin, SGCA encoding for alpha-sarcoglycan, VCP encoding for valosin containing protein, PAX7 encoding for paired box 7, NCAM1 encoding for neural cell adhesion molecule 1 or DMD encoding for dystrophin.

5. The method according to claim 1, wherein the gene editing using CRISPR/Cas-based tools comprises at least one of the following: adenine base editing (ABE), cytidine base editing (CBE/BE), C-to-G base editing (CGBE), glycosylase base editing (GBE), prime editing, non-homologous end joining (NHEJ), microhomology-mediated end joining (MMEJ) and/or homology-directed repair (HDR).

6. The method according to claim 1, wherein the CRISPR/Cas-based gene editing tools are delivered to the cultivated primary muscle stem cells using a plasmid as transport system.

7. The method according to claim 6, herein transfection of the plasmid as transport system for CRISPR/Cas-based gene editing tools was carried out using said human primary muscle cells at a cell density in a range between 40,000 and 90,000 cells/9.5 cm², preferably in a range between 50,000 and 80,000 cells/9.5 cm², such as 55,000 and 75,000 cells/9.5 cm².

8. The method according to claim 15, wherein the CRISPR/Cas-based gene editing tools are delivered to the cultivated primary muscle stem cells using mRNA as transport system.

9. The method according to claim 8, herein transfection of the mRNA as transport system for CRISPR/cas-based gene editing tools was carried out using electroporation (nucleofection).

10. The method according to claim 1, wherein the CRISPR/Cas-based gene editing tools are delivered to the cultivated primary muscle stem cells using recombinant protein as transport system.

11. The method according to claim 10, herein transfection of the recombinant protein as transport system for CRISPR/cas-based gene editing tools was carried out using electroporation (nucleofection).

12. The method according to claim 1, wherein the primary stem cells from said muscle-fiber containing tissue sample are cultivated by a treatment without oxygenation under hypothermic conditions having a defined temperature and a defined atmosphere, wherein the temperature does not exceed 15° C. and the atmosphere has an oxygen content not exceeding 21% (v/v), and wherein the first period of time is 4 days to 4 weeks.

13. The method according to claim 12, herein the temperature does not exceed 10° C. and the oxygen content does not exceed 10% (v/v).

14. The method according to claim 1, wherein genetically modified primary stem cells are further cultivated.

15. (canceled)

16. A genetically repaired human muscle stem cell, wherein it comprises at least one gene encoding for at least one muscle protein, wherein the at least one gene underwent a targeted modification of at least one mutation in said gene.

17. The genetically repaired human muscle stem cell according to claim 16, wherein the at least one modified gene encodes for at least one of the following muscle proteins: LMNA encoding for lamin A/C, CAPN3 encoding for calpain 3, DYSF encoding for dysferlin, SGCA encoding for alpha-sarcoglycan, VCP encoding for valosin containing protein, PAX7 encoding for paired box 7, NCAM1 encoding for neural cell adhesion molecule 1 or DMD encoding for dystrophin.

18. A method for using a genetically repaired human muscle stem cell in cell replacement therapies for muscular dystrophy, in particular all types of limb-girdle muscular dystrophy (LGMD), in particular of type LGMD1/D, LGMD2/R, all X-linked muscular dystrophies (Emery-Dreyfuss MD, Duchenne MD, Becker MD), all MDs caused by repeat expansion (i.e. myotonic dystrophy type 1 and type 2) or repeat deletion (facioscapulohumeral muscular dystrophy) mutations, and Pax7 or VCP myopathy.
